(19) United States
(12) Patent Application Publication
(10) Pub. No.: US 2025/0243444 A1
(43) Pub. Date: Jul. 31, 2025

(54) AN INCUBATOR SYSTEM

(71) Applicant: BPC Instruments AB, LUND (SE)

(72) Inventor: Jing LIU, Lund (SE)

(21) Appl. No.: 18/853,222

(22) PCT Filed: Aug. 10, 2023
(86) PCT No.: PCT/SE2023/050803

§ 371 Date: Oct. 1, 2024

(30) Foreign Application Priority Data
Aug. 16, 2022 (SE) 2250966-5

(51) Int. Cl. C12M 1/00; C12M 1/24; C12M 1/34; C12M 1/36

(52) U.S. Cl. CPC C12M 41/14; C12M 23/08; C12M 41/34; C12M 41/48

(57) ABSTRACT

The present invention describes an incubator system 1 comprisesing—an outer framework (11) with multiple holes (14) into which incubator flasks (20) may be arranged in a vertical position;—an inner space (30) being embedded by an insulating material (34), wherein the outer framework (11) is arranged outside the inner space (30); and—an air cooling and heating unit (40) connected to a PTC element (50) positioned inside the inner space (30).

### FIELD OF THE INVENTION

The present invention relates to an incubator system, especially intended for gas volume or gas flow measurement.

### TECHNICAL BACKGROUND

Different form of incubator systems have been disclosed in the past. Incubator systems have several fields of applications, for example they are used to hold optimal temperature, humidity and other conditions such as the CO₂ and oxygen content for example when growing and maintaining microbiological cultures or cell cultures. When monitoring this type of processes, studies of the yield of biomethane and other biogases can provide important information regarding fundamental evaluation of the possible biogas/methane yield and of the anaerobic biological degradability of organic materials. Also, it provides information about the qualitative appraisal of the kinetics of anaerobic degradation and qualitative evaluation of the inhibitory effect. Other types of gas flows and volume accumulation, e.g. oxygen consumption or evolved carbon dioxide may be of interest when studying other types of processes such as aerobic fermentation and biodegradation processes. Thereby, environments which enable for monitoring accurate gas volume and gas flow measurements, and environments which enables fermentation to be carried out at desired conditions are of greatest importance in these contexts. In order to control the temperature of the incubator, in particular to keep incubation clamber below the environmental temperature, most of the incubator systems known today use compressors for cooling and some type of heating coil or plate for heating. One example is CN215103170 where an incubator for growing microorganisms is disclosed. The incubator is arranged for letting circulating water with a certain temperature regulate the temperature inside the incubator. Another example is disclosed in CN216123655 where a heat exchange assembly is connected to a condenser and a compressor where heat exchange occurs. One of the problems with known techniques is that indirect cooling/heating is used, i.e., the temperature of the targeted area is regulated by surrounding water or air which in turn has been regulated in a second heating or cooling apparatus such as the compressor or heating coil. As can be understood, this solution includes heavy systems with high complexity and cost, high energy consumption and leaves a large ecological footprint. Moreover, the large temperature offset is due to indirect heating/cooling and they are often inconvenient to use. The present invention is directed to providing an incubator system, especially intended for integration in a system for gas volume or/and gas flow measurement, which is user-friendly both to install and to use in an automatic fashion.

### SUMMARY OF THE INVENTION

The stated purpose above is achieved by an incubator system comprising
an outer framework with multiple holes into which incubator flasks may be arranged in a vertical position;
an inner space being embedded by an insulating material, wherein the outer framework is arranged outside the inner space; and
an air cooling and heating unit connected to a PTC element positioned inside the inner space.

The incubator system according to the present invention utilizes thermoelectric cooling and heating using the Peltier effect. Thermoelectric modules use the Peltier effect to create a heat flux at the junction of two different materials, making it a solid-state active heat pump as it transfers heat from one side of the device to the other, with consumption of electrical energy. Depending on the direction of the current at the moment, heat is either transported to or from said side of the device, making it suitable for using for either heating or cooling. This type of device is also known as a Peltier device, Peltier heat pump, solid state refrigerator or thermoelectric cooler (TEC) and occasionally a thermoelectric battery. Using a thermoelectric module for cooling and heating is suitable for the present purpose since this provides an incubator system with a controlled temperature range from 5° C.-60° C. with an average environmental temperature of 20° C. Other advantages obtained through use of thermoelectric modules are that the cooling and heating can easily be switched by altering the polarity of the DC power, and the low power consumption because of the low voltage, typically from 12 DC-48 DC power supply.

The usage of thermoelectric modules for cooling closed spaces is found for example in camping cooling boxes. However, in known techniques the cooling has to be switched on and off manually and no temperature control or precise temperature regulation feature can be found. This can of course lead to fluctuation of the temperature over time and slow feedback which is not suitable in an incubation system where a constant and pre-determined temperature is crucial. Due to this, the incubator system according to the present invention has a PTC element connected to the thermoelectric module which enables automatic regulation of cooling and heating. The PTC element is supplied with a pre-set temperature and enables for precise temperature control which is desirable in an incubator system.

The incubator system according to the present invention also has an inner space being embedded by an insulating material which helps to amplify the precise temperature control together with the PTC element.

The incubator system according to the present invention also have a special design of the chamber, which is well thermo isolated and air sealed, including an outer framework with multiple holes into which incubator flasks may be arranged in a vertical position if the incubator is used for microbial fermentation. Depending on the current use and demand, flasks with different volume may be suspended without dead corners so that ventilated air can evenly distribute cooling and heating inside the thermo chamber in an effective manner.

Even though the intention is to use the incubator system according to the present invention to replace traditional heating/cooling baths it should not be limited thereto, but the concept could advantageously be applied also to wider applications such as a temperature-controlled chamber for various kinds of biological experiments. It may also be used for non-biological applications due to the voluntarily removable flasks. It should also be mentioned here that even other measuring devices can be added depending on the type of application.

To summarize, the present invention provides an incubator system where the cooling and heating is enabled in a simple and automatic way. The existing incubator systems of today operate with indirect cooling and heating, such via the use of a water bath or another form of water heat exchanger solution. As mentioned, this is problematic as it requires more control by operators and it also includes a high system complexity and cost and a high energy consumption. Moreover, it leaves a large ecological footprint due to the heave instrument. The large temperature offset is due to indirect temperature and the instrument is often inconvenient to use. The incubator system according to the present invention also has a very low power consumption, e.g., in a range of only 100-150 W, in comparison to conventional thermo-water or cooling baths which often consume at least 1500-2000 W per unit. Moreover, and because of the convenient and easy handling, the incubator system according to the present system increases the quality and efficiency of the work of the users.

The present invention, however, has included a concept involving insulation and at least one PTC element so that automatic air cooling and heating is enabled in a very quick and simple way.

### Specific Embodiments of the Invention

Below some specific embodiments of the present invention are described further. Moreover, the present invention and aspects thereof are discussed in more detail.

According to one embodiment of the present invention, the PTC element is a single PTC element for both cooling and heating. This is a simple and efficient solution of the concept according to the present invention. In this case, the PTC element is placed vertically against the wall in order to minimize the accumulation of ice and water condensation on the surface of the cooling sink.

According to yet another embodiment, at least some of the multiple holes have different size. This architecture implies that different types of flasks, size-wise and/or in relation to the type of flask, which in turn may be dependent on the intended purpose. To provide some holes with one size and some other ones with another size provides for flexibility for a user so that the incubator system may be used for different flasks. According to one embodiment, flasks with the sizes 18×0.5 L, 18×1 L and 9×2 L may be suspended to the system of the present invention. According to yet another embodiment, a set-up with flaks with the sizes 15×0.5 L, 15×1.0 L and 6×2.0 L may be used instead. As an example mentioned below, incubator flasks may be combined with chemical fixation chambers in an incubator system kit according to the present invention. This system may also be combined with a measuring device or an array of connected measuring devices for gas flow measurement of gas being produced or consumed in the flasks in the incubator system.

Moreover, according to yet another embodiment, the insulating material is arranged to thermally insulate the inner space and also creating the multiple holes. This alternative implies that the insulating material creates and shapes the entire inner space.

According to another specific embodiment, the incubator system comprises wheels on a bottom side to provide for mobility. The wheels ensures that an incubator system according to the present invention is flexible also in terms positioning, which in turn also implies that the system may be used for one thing in one position and something else in another position. Therefore, the system according to the present invention may be used as a mobile incubator station.

According to another specific embodiment, the incubator system comprises a moveable plate for a laptop with connection to gas volume and flow measurement system including embedded microcontroller, such as Gas Endeavour®, BPC® Go, AMPTS®.

Furthermore, according to another embodiment, the incubator system comprises a temperature control unit and a temperature display unit. Moreover, according to yet another embodiment, the temperature control unit is arranged to automatically switch between cooling and heating based on a pre-set temperature. The control unit or system according to the present invention may control the single PTC for cooling and heating according to a desired temperature setting. This will require a function for switching polarization of a DC power supply in order to meet the requirement of automatically switching cooling and heating modes.

Furthermore, the incubator system according to the present invention may also include lighting/illuminating control, moisture control and O₂ or CO₂ control. This may be of interest for different industrial applications, such as simulating photosynthesis of algal cultivation.

The present invention also provides an incubator system kit comprising an incubator system according to the present invention and multiple incubator flasks, preferably where at least some of the multiple incubator flasks are of different types. All flasks are suitably hanged or suspended inside the incubator chambers with a lid that fit well for e.g., a GL45 flask and possible other standard laboratory flasks. In line with this, according to one embodiment, the multiple incubator flasks each comprises a flask body, a hang-on collar unit and a closing lid unit, wherein the hang-on collar unit is fastened by means of the closing lid unit and wherein the hang-on collar unit is arranged to hold a flask in place freely hanging inside of a hole of the incubator system. The lid unit is suitably of a cork type which is closed by screwing.

Moreover, the flasks and/or the outer framework are designed such that an agitation system optionally can be attached thereto.

In relation to the above it should, however, be noted that the incubator may also be used without flasks for non-microbial fermentation related incubation. In that case, the closing lid units may replace the flasks suspended in the holes.

As hinted above, according to one embodiment, the multiple incubator flasks comprises one flask type being a digester flask and another flask type being a chemical fixation chamber, wherein the chemical fixation chambers may be of the type ex-situ chemical flasks or in-situ chemical fixation chambers. The chemical fixation is intended to fixate other gases than biomethane, hydrogen, nitrogen, oxygen, at least CO₂. This is very usable for performing a biomethane potential test and aerobic biodegradability test applications. One great advantage with the use of chemical fixation chambers is that the temperature between the reaction flasks where the fermentation is taking place and the chemical fixation chamber(s) can be evened out, so that less or no water condensation is formed inside, which otherwise could occur due to the temperature drop from the reaction flasks to the chemical fixation chambers. A similar configuration may also apply to in-vitro digestibility (animal feed) application.

Moreover, according to yet another embodiment, the incubator system kit or station also comprises one or more measuring devices, preferably one or more gas volume and flow meters with working principle based on liquid displacement. As mentioned, this may be one measuring device or an array of connected measuring devices. Furthermore, the measuring devices may be gas volume and flow measuring devices. The incubator system kit may also include sensors for pH, gas composition (CH₄, CO₂, O₂, etc), light intensity, etc.

Furthermore, the present invention also refers to use of an incubator system kit according to the present invention, for measuring biomethane gas in a biogas flow, such as for measuring biological methane potential of a biosample in anaerobic digestion processes, for measuring oxygen, carbon dioxide and/or biogas volume and/or flow for measuring aerobic biodegradability of plastics or chemicals, or measuring a mixed gas comprising at least carbon dioxide and/or methane for in-vitro digestibility analysis or application of any other microbial respiration tests.

According to one embodiment of the invention, the incubator system has an integrated place for position one or more gas measuring units, such as BPC® Go or Gas Endeavour® or anything equivalent which accurately can measure low gas volume and flow for any type of gas production or consumption. Together with the whole thermo chamber this can serve as a complete analysis system for biogas, in-vitro digestibility and biodegradability assays and more in other biological fermentation assays. The gas measuring units could provide automatic and simultaneous measurement of gas volume, flow and possible main composition in real-time. Moreover, the units have a modular design, which enables array arrangement and integration with different types of flasks. This part of the process is often located after the chemical fixation where several gases may be eliminated.

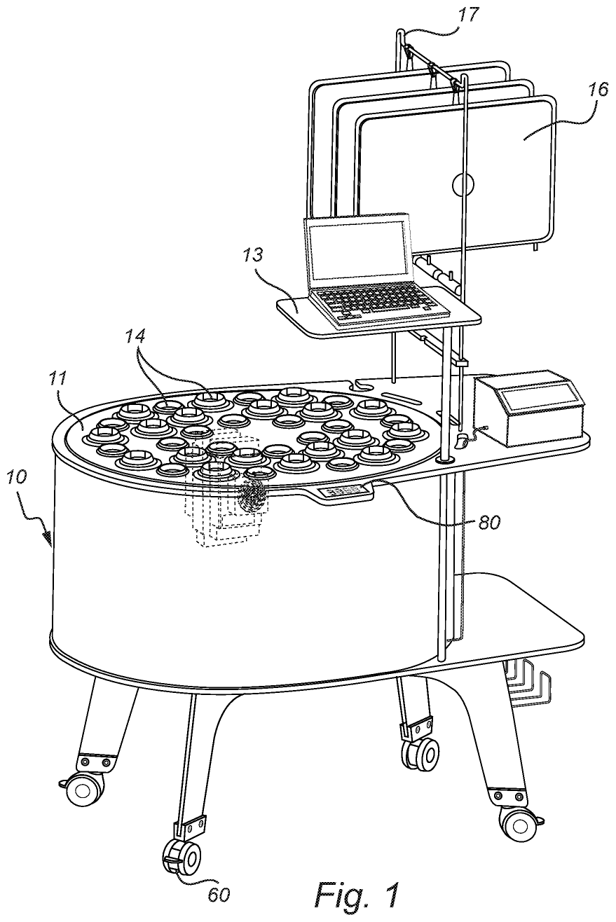

In relation to the drawings, in FIGS. 1-3 there are shown one incubator system according to one embodiment of the present invention. Details are further explained below.

### DETAILED DESCRIPTION OF THE DRAWINGS

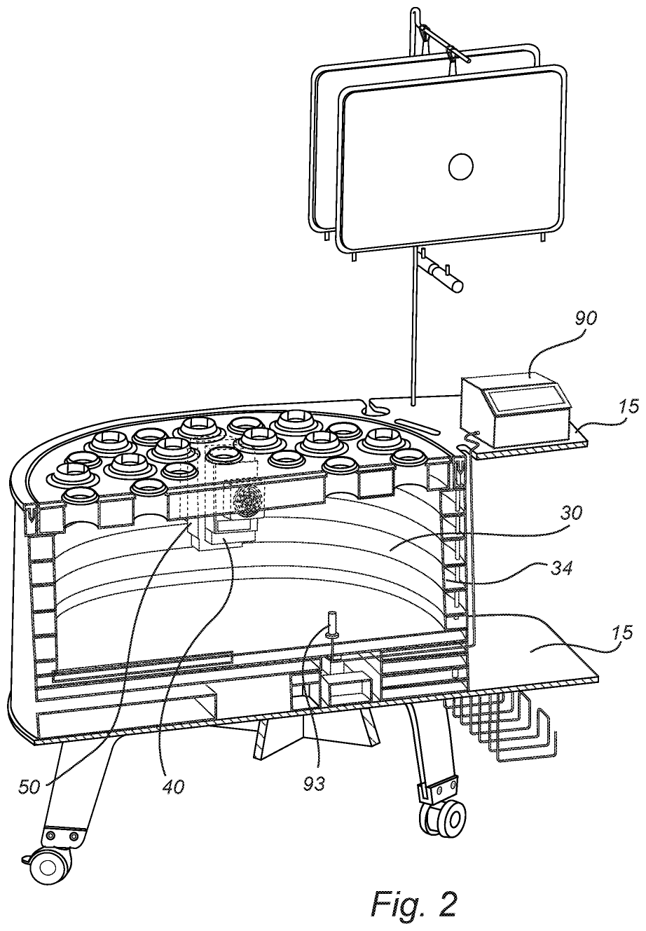

In FIG. 1 there is shown one embodiment of an incubator system 1 according to the present invention. In this case, the incubator system 1 comprises a thermo chamber 10, including an outer framework 11 with multiple holes 14 into which incubator flasks 20 may be arranged in a vertical position. In this case, the incubator system 1 also comprises a frame 17 for holding gas bags 16. As can be seen in FIG. 2, the incubator system 1 has an inner space 30 being embedded by an insulating material 34, and an air cooling and heating unit 40 connected to a PTC element 50 positioned vertically inside the inner space 30. Moreover, in this case the PTC element 50 is a single PTC element for both cooling and heating. As shown in FIG. 1 at least some of the multiple holes 14 have different sizes, enabling for use of different flask sizes.

Furthermore, the isolation material 34 is arranged to thermally insulate the inner space 30 and also creating the multiple holes 14.

As shown in FIG. 1, incubator system 1 comprises wheels 60 on a bottom side to provide for mobility, as a mobile incubator station, and a removable table for a computer 13. Moreover, in this case the incubator system 1 comprises a temperature control unit and a temperature display unit 80. The temperature control unit may be arranged to automatically switch between cooling and heating based on a pre-set temperature.

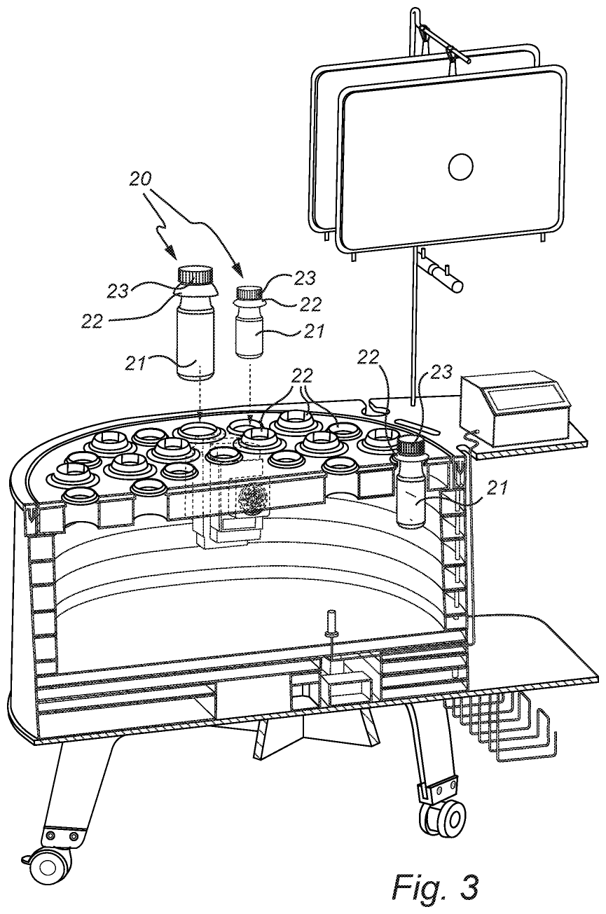

As shown in FIG. 3 an incubator system kit is provided, including the incubator system 1 and multiple incubator flasks 20. The multiple incubator flasks 20 may be of different types, such as digester flask or another flask type being a chemical fixation chamber. However, common to all incubator flasks 20 is that they may comprise a flask body 21, a hang-on collar unit 22 and a closing lid unit 23. The hang-on collar unit is fastened by means of the closing lid unit, and the hang-on collar unit is arranged to hold a flask in place freely hanging inside of a hole of the incubator system 1. Incubator flasks 20 with different volume may be suspended without dead corners so that ventilated air can evenly distribute cooling and heating inside the thermo chamber in an effective manner.

Moreover, the incubator system 1 may also comprise one or more measuring devices 90, preferably one or more gas volume and flow meters with working principle based on liquid displacement. The measuring device 90 may include gas volume and flow measuring devices. The incubator system may also comprise an area 15 intended for placing of the measuring device. The incubator system kit may also include sensors 93 for temperature, pH, gas composition (CH₄, CO₂, O₂, etc.), light intensity, etc.

According to one example of the present invention, a prototype of the incubator system 1 has been developed. In this prototype a reactor for holding 15 pieces 1 L air chambers with cooling capacity of 100 W was tested together with a preferred thermo isolation material. It turned out that the pre-set temperature between 5° C. to 60° C. was held constant over an extensive incubation time period, and could easily be regulated just as desired.

1. An incubator system comprising
an outer framework with multiple holes into which incubator flasks may be arranged in a vertical position;
an inner space being embedded by an insulating material, wherein the outer framework is arranged outside the inner space; and
an air cooling and heating unit connected to a PTC element positioned inside the inner space.

2. The incubator system according to claim 1, wherein the PTC element is a single PTC element for both cooling and heating.

3. The incubator system according to claim 1, wherein at least some of the multiple holes have different size.

4. The incubator system according to claim 1, wherein the insulating material is arranged to thermally insulate the inner space and also creating the multiple holes.

5. The incubator system according to claim 1, wherein the incubator system comprises wheels on a bottom side to provide for mobility.

6. The incubator system according to claim 1, wherein the incubator system comprises a temperature control unit and a temperature display unit.

7. The incubator system according to claim 6, wherein the temperature control unit is arranged to automatically switch between cooling and heating based on a pre-set temperature.

8. An incubator system kit comprising an incubator system according to claim 1 and multiple incubator flasks, preferably where at least some of the multiple incubator flasks are of different types.

9. The incubator system kit according to claim 8, wherein the multiple incubator flasks each comprises a flask body, a hang-on collar unit and a closing lid unit, wherein the hang-on collar unit is fastened by means of the closing lid unit and wherein the hang-on collar unit is arranged to hold a flask in place freely hanging inside of a hole of the incubator system.

10. The incubator system kit according to claim 8, wherein the multiple incubator flasks comprises one flask type being a digester flask and another flask type being a chemical fixation chamber.

11. The incubator system kit according to claim 8, wherein the incubator system also comprises one or more measuring devices, preferably one or more gas volume and flow meters with working principle based on liquid displacement.

12. A method comprising using of an incubator system kit according to claim 8, for measuring biomethane gas in a biogas flow, such as for measuring biological methane potential of a biosample in anaerobic digestion processes, for measuring oxygen, carbon dioxide and/or biogas volume and/or flow for measuring aerobic biodegradability of plastics or chemicals, or measuring a mixed gas comprising at least carbon dioxide and/or methane for in-vitro digestibility analysis or application of any other microbial respiration tests.
